(19) United States
(12) Patent Application Publication
(10) Pub. No.: US 2025/0295518 A1
(43) Pub. Date: Sep. 25, 2025

(54) FLUID COLLECTION DEVICES AND METHODS OF USING THE SAME

(71) Applicant: PUREWICK CORPORATION, Covington, GA (US)

(72) Inventor: Kathryne Rose Eckert, Acworth, GA (US)

(21) Appl. No.: 19/179,540

(22) Filed: Apr. 15, 2025

Related U.S. Application Data
(63) Continuation of application No. 16/904,868, filed on Jun. 18, 2020, now Pat. No. 12,295,876.
Application No. 16/904,868 is a continuation of application No. PCT/US2019/029614, filed on Apr. 29, 2019.
(60) Provisional application No. 62/665,317, filed on May 1, 2018.

(51) Int. Cl. A61F 5/455; A61F 5/44; A61M 1/00

(52) U.S. Cl. CPC A61F 5/4556; A61F 5/44; A61F 5/4401; A61M 1/962; A61M 2202/0496; A61M 2210/1092

(57) ABSTRACT

In an example, a fluid collection device includes a fluid impermeable barrier that at least partially defines a chamber. The fluid impermeable barrier also defines an opening extending therethrough. The opening is configured to be positioned adjacent to a female urethra. The fluid collection device also includes a conduit at least partially disposed in the chamber. The conduit defines at least one aperture that provides fluid communication between an interior of the conduit and the chamber. The conduit also includes an outlet that provides fluid communication between the interior of the conduit and a vacuum source or a fluid storage container. The size and shape of the fluid collection device helps minimize the area of a user's skin both that contacts the device and to which a vacuum pressure is applied.

### CROSS-REFERENCE TO RELATED APPLICATIONS

This application is a continuation of U.S. application Ser. No. 16/904,868 filed Jun. 18, 2020, which is a continuation of PCT International Application No. PCT/US2019/029614 filed Apr. 29, 2019, which application claims priority from U.S. Provisional Application No. 62/665,317 filed on May 1, 2018, the disclosure of each of which are incorporated herein, in their entirety, by this reference.

### BACKGROUND

An individual may have limited or impaired mobility such that typical urination processes are challenging or impossible. For example, the individual may have a disability, medical condition, or have undergone a surgical procedure that impairs mobility. In another example, the individual may have restricted access to a restroom or experience other movement restrictions such as those experienced by pilots, drivers, and workers in hazardous or remote areas. Additionally, fluid collection from the individual may be needed for monitoring purposes or clinical testing.

Bed pans and urinary catheters, such as Foley catheters, can be used to address some of these situations. However, bed pans and urinary catheters have several problems associated therewith. For example, bed pans can be prone to spills and can cause discomfort and other hygiene issues. Urinary catheters be can be uncomfortable, painful, difficult to self-insert, and can cause urinary tract infections. For women, the urogenital tissue is delicate and prone to damage by contact with some existing fluid collection devices.

Thus, users and manufacturers of fluid collection devices continue to seek new and improved devices, systems, and methods to collect urine.

### SUMMARY

In an example, a fluid collection device includes a fluid impermeable barrier that at least partially defines a chamber. The fluid impermeable barrier also defines an opening extending therethrough. The opening is configured to be positioned adjacent to a female urethra. The fluid collection device also includes a conduit at least partially disposed in the chamber. The conduit defines at least one aperture that provides fluid communication between an interior of the conduit and the chamber. The conduit also includes an outlet that provides fluid communication between the interior of the conduit and a vacuum source or a fluid storage container. The size and shape of the fluid collection device helps minimize the area of a user's skin both that contacts the device and to which a vacuum pressure is applied.

In an embodiment, a method to collect fluid includes positioning a fluid collection device adjacent to a female perineum such that an opening defined by a fluid impermeable barrier of the fluid collection device covers a urethral opening, and such that at least one of a clitoris, a mons pubis, a vaginal opening, an anal aperture, or an intergluteal cleft are free from coverage by the fluid collection device. The method also includes receiving fluid from the female urethra into a chamber of the fluid collection device, the chamber of the fluid collection device at least partially defined by the fluid impermeable barrier.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a schematic top isometric view of a fluid collection device, according to an embodiment.

FIG. 1B is a schematic cross-sectional view of the fluid collection device of FIG. 1A.

FIG. 2 is a schematic of a fluid collection system, according to an embodiment.

FIG. 3 is a flow diagram of a method to use any of the fluid collection devices and/or fluid collection systems disclosed herein, according to an embodiment.

### DETAILED DESCRIPTION

Embodiments disclosed herein are related to devices, systems, and methods of using fluid collection devices. In an embodiment, a fluid collection device includes a fluid impermeable barrier that at least partially defines a chamber. The fluid impermeable barrier also defines an opening extending therethrough. The opening is configured to be positioned adjacent to a female urethra. The fluid collection device also includes a conduit at least partially disposed in the chamber. The conduit defines at least one aperture that provides fluid communication between an interior of the conduit and the chamber. The conduit also includes an outlet that provides fluid communication between the interior of the conduit and a vacuum source or a fluid storage container. The size and shape of the fluid collection device helps minimize the area of a user's skin both that contacts the device and to which a vacuum pressure is applied.

The fluid collection devices disclose herein are configured to collect fluids from a user. The fluids collected by the fluid collection devices include urine. The fluids collected by the fluid collection devices can also include vaginal discharge, penile discharge, reproductive fluids, blood, sweat, or other bodily fluids.

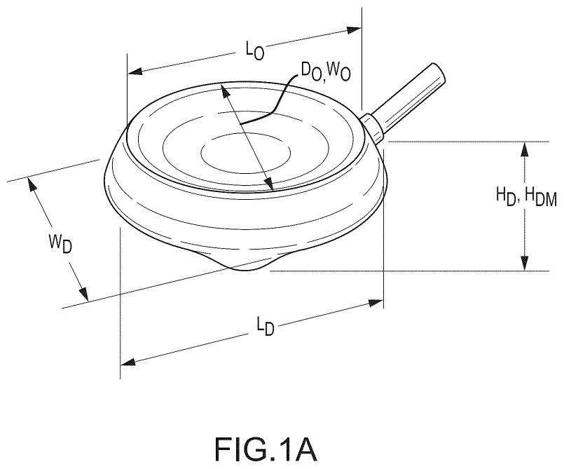

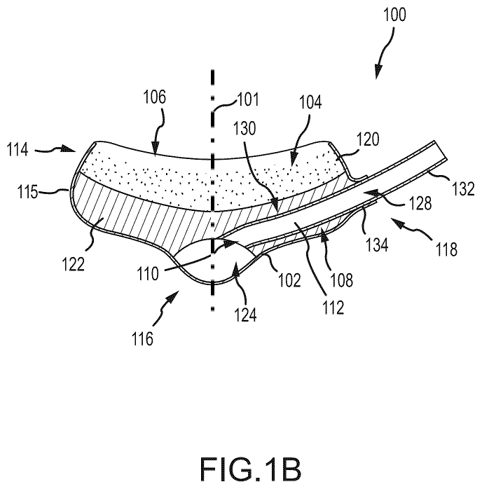

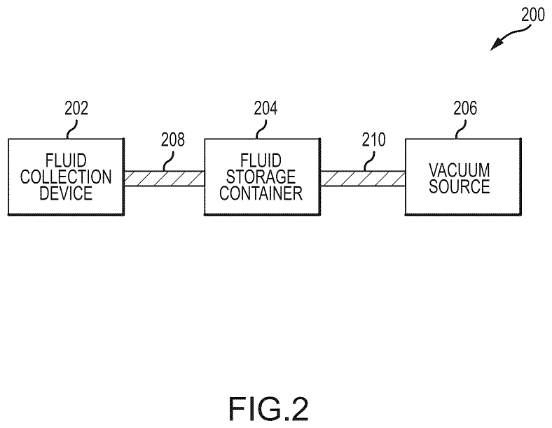

FIG. 1A is a schematic top isometric view and FIG. 1B is a cross-sectional view of a fluid collection device 100, according to an embodiment. The fluid collection device 100 is an example of a fluid collection device 100 that is configured to receive fluids, such as urine, from a female. The fluid collection device 100 includes a fluid impermeable barrier 102. The fluid impermeable barrier 102 at least partially defines a chamber 104 and an opening 106. The opening 106 extends through the fluid impermeable barrier 102 thereby enabling fluids to enter the chamber 104. The opening 106 is configured to be positioned adjacent to a female urethra. The fluid collection device 100 also includes a conduit 108 (e.g., tube) that is at least partially disposed in the chamber 104. The conduit 108 defines at least one aperture 110 therein that provides fluid communication between an interior 112 of the conduit 108 and the chamber 104. For example, the conduit 108 may define at least one aperture 110 therein that either directly or indirectly fluidly couples the interior 112 of the conduit 108 to the chamber 104. The conduit 108 also includes an outlet 128 that is configured to provide fluid communication between at least one of a vacuum source (e.g., vacuum source 206 of FIG. 2) or a fluid collection container (e.g., fluid collection container 204 of FIG. 2). Accordingly, the outlet 128 may be fluidly coupleable to at least one of the vacuum source 206 or the fluid collection container 204.

The fluid collection device 100 is configured to receive the fluids into the chamber 104 via the opening 106. The opening 106 traverses up to 100% of an upper portion 114 of the fluid collection device 100. For example, the opening 106 can traverse about 75% to about 100%, about 80% to about 100%, about 85% to about 100%, about 75% to about 95%, or about 75% to about 90% of the upper portion 114. The opening 106 can exhibit a generally circular or oblong shape that is configured to be positioned immediately beneath the urethral opening such that it is approximately centered beneath the urethral opening. The opening 106 can exhibit a diameter (D_{O}) or other maximum dimension to cover the urethral opening when in use, while large areas of surrounding perineal skin or tissue are free from coverage or left uncovered by the fluid collection device 100. For example, any one or more of the clitoris, mons pubis, vaginal opening, anal aperture, or intergluteal cleft may be free from coverage by the fluid collection device 100 or left uncovered by the fluid collection device 100 when the fluid collection device is in use. To cover the urethral opening while large areas of surrounding perineal skin or tissue are free from coverage or left uncovered by the fluid collection device 100, the opening 106 can exhibit a diameter (D_{O}) or other maximum dimension of about 0.25 inch to about 1.0 inch, about 0.4 inch to about 0.9 inch, about 0.5 inch to about 0.8 inch, about 0.6 inch to about 0.7 inch, about 0.4 inch to about 0.6 inch, 0.5 inch to about 0.7 inch, 0.6 inch to about 0.8 inch, 0.7 inch to about 0.9 inch, about 0.25 inch to about 0.4 inch, about 0.4 inch to about 0.5 inch, about 0.5 inch to about 0.6 inch, about 0.6 inch to about 0.7 inch, about 0.7 inch to about 0.8 inch, about 0.8 inch to about 0.9 inch, about 0.9 inch to about 1.0 inch, less than about 1.0 inch, less than about 0.9 inch, less than about 0.8 inch, less than about 0.7 inch, less than about 0.6 inch, less than 0.5 inch, less than about 0.4 inch, or less than about 0.3 inch.

To cover the urethral opening while large areas of surrounding perineal skin or tissue are free from coverage or left uncovered by the fluid collection device 100, the opening can exhibit a width (W_{O}) of about 0.25 inch to about 1.0 inch, about 0.4 inch to about 0.9 inch, about 0.5 inch to about 0.8 inch, about 0.6 inch to about 0.7 inch, about 0.4 inch to about 0.6 inch, 0.5 inch to about 0.7 inch, 0.6 inch to about 0.8 inch, 0.7 inch to about 0.9 inch, about 0.25 inch to about 0.4 inch, about 0.4 inch to about 0.5 inch, about 0.5 inch to about 0.6 inch, about 0.6 inch to about 0.7 inch, about 0.7 inch to about 0.8 inch, about 0.8 inch to about 0.9 inch, about 0.9 inch to about 1.0 inch, less than about 1.0 inch, less than about 0.9 inch, less than about 0.8 inch, less than about 0.7 inch, less than about 0.6 inch, less than 0.5 inch, less than about 0.4 inch, or less than about 0.3 inch.

To cover the urethral opening while large areas of surrounding perineal skin or tissue are free from coverage or left uncovered by the fluid collection device 100, the opening can exhibit a length (L_{O}) of about 0.5 inch to about 1.75 inches, about 0.5 inch to about 1.5 inches, about 1.0 inch to about 1.75 inches, about 0.75 inch to about 1.5 inches, about 0.5 inch to about 1.0 inch, about 0.5 inch to about 0.75 inch, about 0.75 inch to about 1.0 inch, about 1.0 inch to about 1.25 inches, about 1.25 inches to about 1.50 inches, about 1.50 inches to about 1.75 inches, less than about 1.75 inches, less than about 1.5 inches, less than about 1.25 inches, less than about 1.0 inch, less than about 0.75 inch, or less than 0.5 inch.

In some embodiments, the opening 106 may be vertically oriented (e.g., having a major axis parallel to the longitudinal axis 101 of the device 100). In some embodiments, (not shown), the opening 106 may be horizontally oriented (e.g., having a major axis perpendicular to the longitudinal axis 101 of the device 100). In an example, the fluid impermeable barrier 102 can be configured to be attached to the user, such as adhesively attached (e.g., with a hydrogel adhesive) to the user. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 is also configured to temporarily store the fluids in the chamber 104. For example, the fluid impermeable barrier 102 can be formed of any suitable fluid impermeable materials, such as a fluid impermeable polymer (e.g., silicone, polypropylene, polyethylene, polyethylene terephthalate, a polycarbonate, etc.), a metal film, another suitable material, or combinations thereof. As such, the fluid impermeable barrier 102 substantially prevents the fluids from exiting the portions of the chamber 104 that are spaced from the opening 106. In an example, the fluid impermeable barrier 102 can be air permeable and fluid impermeable. In such an example, the fluid impermeable barrier 102 can be formed of a hydrophobic material that defines a plurality of pores. In an example, one or more portions of the outer surface of the fluid impermeable barrier 102 can be formed from a soft and/or smooth material thereby reducing chafing of the skin or tissue with which the barrier 102 comes into contact. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 can include a fluid permeable membrane 120 disposed in the chamber 104. The fluid permeable membrane 120 can cover at least a portion (e.g., all) of the opening 106. That is, an outer surface of the fluid permeable membrane 120 may be free from coverage by another layer of material. The outer surface of the fluid permeable membrane 120 may be substantially flush with terminating ends of side walls of the fluid impermeable barrier 102. The fluid permeable membrane 120 can be configured to wick any fluid away from the opening 106 thereby preventing the fluid from escaping the chamber 104. The permeable properties referred to herein can be wicking, capillary action, diffusion, or other similar properties or processes, and are referred to herein as “permeable” and/or “wicking.” Such “wicking” may occur absent of absorption into the wicking material. The fluid permeable membrane 120 can include a one-way fluid movement fabric. As such, the fluid permeable membrane 120 can remove fluid from the area around the female urethra, thereby leaving the urethra dry. The fluid permeable membrane 120 can also wick the fluid generally towards an interior of the chamber 104, such as a reservoir 124 (shown in FIG. 1). The fluid permeable membrane 120 can include any material that can wick the fluid. For example, the fluid permeable membrane 120 can include fabric, such as a gauze (e.g., a silk, linen, or cotton gauze), another soft fabric, or another smooth fabric. Forming the fluid permeable membrane 120 from gauze, soft fabric, and/or smooth fabric can reduce chafing caused by the fluid collection device 100.

In some embodiments, the fluid permeable membrane 120 can include a porous or fibrous material, such as hydrophilic polyolefin. In some embodiments, the fluid permeable membrane 120 consists of or consists essentially of a porous or fibrous material, such as hydrophilic polyolefin. Examples of polyolefin that can be used in the fluid permeable membrane 120 include, but are not limited to, polyethylene, polypropylene, polyisobutylene, ethylene propylene rubber, ethylene propylene diene monomer, or combinations thereof. The porous or fibrous material can be extruded into a shape to fit within the chamber 106 of the fluid impermeable barrier 102. The fluid permeable membrane 120 can include varying densities or dimensions. Moreover, the fluid permeable membrane 120 can be manufactured according to various manufacturing methods, such as molding, extrusion, or sintering.

The fluid collection device 100 can include a fluid permeable support 122 disposed in the chamber 104. The fluid permeable support 122 is configured to support the fluid permeable membrane 120 when the fluid permeable membrane 120 is formed from a foldable, flimsy, or otherwise easily deformable material. For example, the fluid permeable support 122 can be positioned such that the fluid permeable membrane 120 is disposed between the fluid permeable support 122 and the fluid impermeable barrier 102. As such, the fluid permeable support 122 can support and maintain the position of the fluid permeable membrane 120. The fluid permeable support 122 can be formed from any fluid permeable material that is less deformable than the fluid permeable membrane 120. For example, the fluid permeable support 122 can include spun plastic fibers. In an example, the fluid permeable support 122 can be omitted from the fluid collection device 100.

In some examples, the fluid permeable membrane 120, the fluid permeable support 122, and at least a portion of the conduit 108 together can at least substantially completely fill the chamber 104. In another example, the fluid permeable membrane 120, the fluid permeable support 122, and at least a portion of the conduit 108 do not substantially completely fill the chamber 104. In such an example, the fluid collection device 100 includes a reservoir 124 disposed in the chamber 104. The reservoir 124 is a substantially unoccupied portion of the chamber 104 that is void of other materials. Fluids are wicked away from the opening by the fluid membrane 120 and/or fluid permeable support 122 to the reservoir 124. The reservoir 124 can store at least some of the fluids therein for removal from the chamber 104 through a conduit 108. In these and other embodiments, the fluid permeable membrane 120, the fluid permeable support 122, at least a portion of the conduit 108, and the reservoir 124 can at least substantially completely fill the chamber 104.

In an example, the reservoir 124 can be located in a portion of the chamber 104 that is closest to the aperture 110. The reservoir 124 can be located in a portion of the chamber 104 distal from the opening 106. However, the reservoir 124 can be located at different locations in the chamber 104. For example, the reservoir 124 can be located in a portion of the chamber 104 that is closest to the outlet 128. In another example, fluid collection device 100 can include multiple reservoirs, such as a first reservoir that is located in a portion of the chamber 104 that is closest to the aperture 110 and a second reservoir that is located in a portion of the chamber 104 that is closest to the outlet 128. In another example, the fluid permeable support 122 is spaced from at least a portion of the conduit 108 and the reservoir 124 can be the space between the fluid permeable support 122 and the conduit 108. In an example, and with reference to FIG. 1B, the fluid permeable membrane 120 can be arranged within the chamber such that it is positioned below and adjacent to the opening 106 to remove fluid from the area around the female urethra, thereby leaving the urethra dry. The fluid permeable membrane 120 can be positioned above and adjacent to the fluid permeable support 122. The fluid permeable support 122 can be positioned between the fluid permeable membrane 120 and the reservoir 124. The fluid permeable support 122 can be positioned around at least a portion of the conduit 108. The arrangement of the opening 106, fluid permeable membrane 120, fluid permeable support 122, and reservoir 124 can help direct fluid towards the reservoir 124 and/or towards the conduit 108.

In other embodiments, the fluid permeable membrane 120 includes a singular and porous body, and the fluid collection device 100 does not include a fluid permeable support 122. That is, during use, the fluid permeable membrane 120 extends continuously from the opening 106 to the reservoir 124 to wick any fluid from the opening 106 directly to the reservoir 122. In these and other embodiments, the fluid permeable membrane, at least a portion of the conduit 108, and the reservoir 120 can at least substantially completely fill the chamber 104. A singular fluid permeable membrane 120 is advantageous to conventional systems, which typically require an air-laid nonwoven pad covered by a ribbed fabric compression bandage, because a singular fluid permeable membrane 120 reduced the number of components in the fluid collection device 100, reduces the assembly time of the fluid collection device 100, requires shelf-life data for only a single component, and provides a latex-free single component. Moreover, as the fluid impermeable barrier is flexible and the fluid permeable membrane 120 is configured to wick fluid from the body rather than absorb fluid from the body and hold the fluid against the body, the fluid collection device 100, in many embodiments, is free from a seal or cushioning ring on an inward edge defining the opening 106.

Other examples of fluid impermeable barriers, fluid permeable membranes, fluid permeable supports, and chambers are disclosed in U.S. patent application Ser. No. 15/260,103 filed on Sep. 8, 2016, the disclosure of which is incorporated herein, in its entirety, by this reference.

The conduit 108 includes and extends between at least one aperture 110 and an outlet 128 that is couplable to, for example, a vacuum source or a fluid storage container. In some examples, the conduit 108 may include one or more portions that are resilient, such as to by having one or more of a diameter or wall thickness that allows the conduit to be flexible. In some examples, the conduit may be frosted or opaque (e.g., black) to obscure visibility of the fluids therein. The aperture 110 provides fluid communication between the interior 112 of the conduit 108 and the chamber 104. For example, the aperture 110 may directly or indirectly fluidly couple the interior 112 of the conduit 108 to the chamber 104 to allow a fluid to flow from the chamber 104 to the interior 112 of the conduit 108. In an embodiment, the aperture 110 can be disposed in or adjacent to the reservoir 124 thereby reducing or preventing pooling of the fluids in the reservoir 124. In an embodiment, the aperture 110 can be located at or near a gravimetrically low point of the fluid collection device 100 thereby preventing pooling of the fluids at the gravimetrically low point of the fluid collection device 100. In an embodiment, the aperture 110 can be spaced apart from the reservoir 124, such as adjacent to the fluid permeable membrane 120 and/or the fluid permeable support 122 thereby preventing fluid stagnation in the fluid permeable membrane 120 and/or the fluid permeable support 122. In the embodiment depicted in FIG. 1B, the conduit 108 includes one aperture 110. In other embodiments, the conduit 108 can include a plurality of apertures 110. In such embodiments, the conduit 108 can include at least one aperture 110 disposed in or near the reservoir 124 and at least one aperture 100 spaced from the reservoir 124. In some embodiments, the hole 134 may be disposed in a region nearer the reservoir 124. In such embodiments, the conduit 108 may be disposed in only the region nearer the reservoir with the inlet 110 being disposed in the end region (e.g., the reservoir 122).

The conduit 108 may include a first portion 130 that is disposed in the chamber 104 and a second portion 132 that is positioned external to the chamber 104. The fluid impermeable barrier 102 can define a hole 134 that allows the conduit 108 to extend therethrough. The first portion 130 of the conduit 108 can define the aperture 110 and the second portion 132 can define the outlet 128. The outlet 128 is configured to establish fluid communication, directly or indirectly, with at least one of the vacuum source or the fluid storage container. For example, the outlet 128 may be fluidly coupled, directly or indirectly, to at least one of the vacuum source or the fluid storage container. In some embodiments, the conduit 108 is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

In an example, one or more components of the fluid collection device 100 can include an antimicrobial material, such as an antibacterial material where the fluid collection device may contact the wearer or the bodily fluid of the wearer. The antimicrobial material can include an antimicrobial coating, such as a nitrofurazone or silver coating. The antimicrobial material can inhibit microbial growth, such as microbial growth due to pooling or stagnation of the fluids. In an example, one or more components (e.g., impermeable barrier 102, conduit 108, etc.) of the fluid collection device 100 can include an odor blocking or absorbing material such as a cyclodextrine containing material or a thermoplastic elastomer (TPE) polymer.

In the design and use of the fluid collection device 100, contact between the fluid collection device 100 and the user is minimized, which can help prevent damage or irritation to the user's perineal skin and tissue, which is often delicate and sensitive. Minimizing the contact between the fluid collection device 100 and the user can be more comfortable for the user compared to devices that contact a larger area of skin or tissue. The fluid collection device 100 can be shaped and sized to cover the urethral opening when in use while large areas of surrounding perineal skin or tissue are free from coverage by the fluid collection device 100 or left uncovered by the fluid collection device 100. For example, any one or more of the clitoris, mons pubis, vaginal opening, anal aperture, or intergluteal cleft may be free from coverage by the fluid collection device 100 or left uncovered by the fluid collection device 100 when the fluid collection device 100 is in use. In the design and use of the fluid collection device 100, suction can be applied to a limited and targeted area, which can also help prevent discomfort and damage or irritation to the user's perineum, including the urogenital skin and tissue. The presently disclosed fluid collection device 100 may be sized smaller than known devices, which have a larger surface area for wicking fluid and a larger volume for storing fluid, but can cause discomfort and damage or irritation to the user's perineum.

In some examples, the fluid collection device 100 is substantially disc-shaped. In these and other embodiments, the fluid impermeable barrier 102 may taper or narrow towards the opening 106. That is, a disc-shaped fluid collection device 100 can include an upper portion 114, a lower portion 116, and an intermediary portion 115 between the upper portion 114 and the lower portion 116. The upper portion 114 extends from the opening 106 to the intermediary portion 115 and can have a smaller diameter or circumference than the intermediary portion 115. In many embodiments, the upper portion 114 can extend to a distance from the opening 106 that is substantially equal to a depth or thickness of the fluid permeable membrane 120 positioned within the chamber 104 of the fluid collection device 100. For example, the upper portion 114 can extend from the opening 106 towards the intermediary portion 115 until the fluid impermeable barrier 102 is free from contact with the fluid permeable membrane 120. In some embodiments, the upper portion 114 can extend only partially from the opening to a portion of the fluid impermeable barrier having the greatest diameter, width, or length. The intermediary portion 115 may extend from the upper portion 114 to the lower portion 116. In some embodiments, the intermediary portion 115 can extend from the upper portion 114 towards the lower portion 116 until fluid impermeable barrier 102 is free from contact with the fluid permeable support 122. In addition to being positioned between the upper portion 114 and the lower portion 116, the intermediary portion may include a greater maximum diameter or circumference than both the upper portion 114 and the lower portion 116. A front portion 118 of the fluid collection device 100, such as the portion at or near the hole 134 through which the conduit 108 extends, can extend laterally away from the device 100. The extended front portion 118 can help provide a smooth and secure interface between the conduit 108 and the fluid impermeable barrier 102. A lower portion 116 of the fluid collection device 100, such as the portion at or near the reservoir 124, can extend longitudinally away from the device 100 along longitudinal axis 101. For example, the lower portion 116 can include a bulbous portion extending from the fluid collection device 100. In some embodiments, the lower portion 116 can extend from the intermediary portion where the fluid impermeable barrier 102 is free from contact with the fluid permeable support 122. The extended lower portion 116 can help provide a region in which fluid can be collected.

The fluid collection device 100 can have width (W_{D}) of about 0.5 inch to about 1.0 inch, about 0.75 inch to about 1.0 inch, about 0.5 inch to about 0.8 inch, about 0.6 inch to about 0.9 inch, about 0.5 inch to about 0.6 inch, about 0.6 inch to about 0.7 inch, about 0.7 inch to about 0.8 inch, about 0.8 inch to about 0.9 inch, about 0.9 inch to about 1.0 inch, less than 1.0 about inch, less than about 0.9 inch, less than about 0.8 inch, less than about 0.7 inch, less than about 0.6 inch, or less than about 0.5 inch. The fluid collection device 100 can have a length (L_{D}) of about 0.5 inch to about 1.75 inches, about 0.5 inch to about 1.5 inches, about 1.0 inch to about 1.75 inches, about 0.75 inch to about 1.5 inches, about 0.5 inch to about 1.0 inch, about 0.5 inch to about 0.75 inch, about 0.75 inch to about 1.0 inch, about 1.0 inch to about 1.25 inches, about 1.25 inches to about 1.50 inches, about 1.50 inches to about 1.75 inches, less than about 1.75 inches, less than about 1.5 inches, less than about 1.25 inches, less than about 1.0 inch, less than about 0.75 inch, or less than 0.5 inch.

The fluid collection device 100 can have a height (H_{D}) of about 0.1 inch to about 0.5 inch, about 0.1 inch to about 0.4 inch, about 0.2 inch to about 0.5 inch, about 0.1 inch to about 0.3 inch, about 0.2 inch to about 0.4 inch, about 0.3 inch to about 0.5 inch, about 0.1 inch to about 0.2 inch, about 0.2 inch to about 0.3 inch, about 0.3 inch to about 0.4 inch, about 0.4 inch to about 0.5 inch, less than 0.5 inch, less than 0.4 inch, less than 0.3 inch, or less than 0.2 inch. The fluid collection device 100 can have a uniform height or its height can vary across the length or width of the device 100. For example, the fluid collection device 100 can have a greatest or maximum height (H_{DM}) from the upper portion 114 to the lower portion 116 of the device 100 when the reservoir 124 is positioned opposite from and distal to the opening 106.

FIG. 2 is a schematic illustration of a fluid collection system 200, according to an embodiment. The system 200 includes a fluid collection device 202. The fluid collection device 202 can be the same as or substantially similar to the fluid collection device 100 of FIG. 1A. The system 200 also include a fluid storage container 204 that is in fluid communication with the fluid collection device 202 via a first tube 208. For example, the fluid storage container 204 may be directly or indirectly fluidly coupled to the fluid collection device 202 via the first tube 208. The system 200 also includes a vacuum source 206 that is in fluid communication with the fluid storage container 204 via a second tube 210. As such, the vacuum source 206 has indirect fluid communication with the fluid collection device 202 via the first and second tubes 208, 210 and the fluid storage container 204. For example, the vacuum source 206 may be indirectly fluidly coupled to the fluid collection device 202 via the first and second tubes 208, 201 and the fluid storage container 204. During operation, the vacuum source 206 can supply a vacuum to the fluid collection device 202.

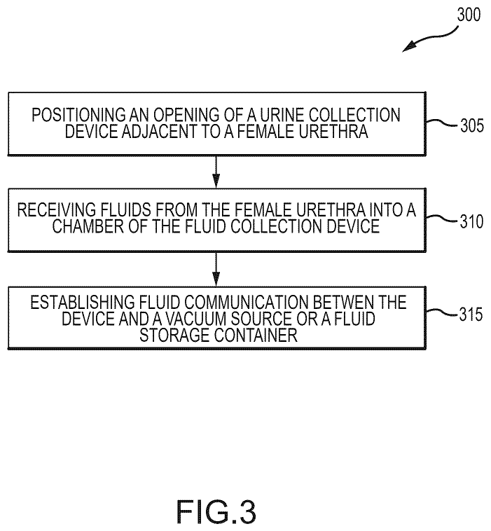

FIG. 3 is a flow diagram of a method 300 to use any of the fluid collection devices and/or fluid collection systems disclosed herein, according to an embodiment. The method 300 can include act 305, which recites “positioning an opening of a urine collection device adjacent to a female urethra.” Act 305 may be followed by act 310, which recites “receiving fluids from the female urethra into a chamber of the fluid collection device.” Act 310 may be followed by act 315, which recites “establishing fluid communication between the device and a vacuum source or a fluid storage container.”

Acts 305, 310, and 315 of the method 300 are for illustrative purposes. For example, the act 305, 310, and 315 of the method 300 can be performed in different orders, split into multiple acts, modified, supplemented, or combined. In one example, act 315 of the method 300 can be omitted from the method 300.

Act 305 recites “positioning an opening of a fluid collection device adjacent to a female urethra.” In an example, act 305 can include positioning the opening of a female fluid collection device such that the fluid permeable membrane of the female fluid collection device abuts or is positioned proximate to the female urethra.

Act 310 recites “receiving fluids from the female urethra into a chamber of the fluid collection device.” For example, act 310 can include wicking the fluids away from the opening using a fluid permeable membrane and a fluid permeable support. Act 310 can include flowing the fluid towards a portion of the chamber that is in fluid communication with an aperture of a conduit. For instance, act 310 can include flowing the fluids to a substantially unoccupied portion of the chamber (e.g., a reservoir) or to a gravimetrically low point of the chamber.

Optional act 315 recites “establishing fluid communication between the device and a vacuum source or a fluid storage container.” A fluid communication can permit transferring fluids to another location or collecting fluids in a receptacle such as a fluid storage container. Establishing fluid communication may include fluidly connecting the device to the vacuum source or the fluid storage container. Act 315 can include the transfer and/or collection of fluids.

While various aspects and embodiments have been disclosed herein, other aspects and embodiments are contemplated. The various aspects and embodiment disclosed herein are for purposes of illustration and are not intended to be limiting.

1. A female urine collection device, comprising:
a fluid impermeable material at least partially defining a chamber and an opening in fluid communication with the chamber and configured to be positioned adjacent to a female urethral opening; and
one or more fluid permeable members disposed in the chamber at least proximate to the opening, at least a portion of the one or more fluid permeable members being positioned on the female urine collection device to directly receive urine discharged from a female user; and
wherein the fluid impermeable material has a width of about 0.5 inch to about 1.0 inch and a length of about 0.5 inch to about 0.75 inch such that when the opening is covering a urethral opening of the female user, the perineum of the female user is generally free from coverage by the female urine collection device.

2. The female urine collection device of claim 1, wherein:
a first side of the fluid impermeable material defines the opening;
a second side of the fluid impermeable material at least partially defines the chamber opposite to the opening;
at least the portion of the one or more fluid permeable members positioned to directly receive the urine discharged from the female user is positioned between the first side and the second side of the fluid impermeable material.

3. The female urine collection device of claim 2, wherein the one or more permeable members include at least a fluid permeable membrane, at least a portion of the fluid permeable membrane being positioned between the first side and the second side of the fluid impermeable material to directly receive the urine discharged from the female user.

4. The female urine collection device of claim 3, wherein the one or more permeable members include a plurality of permeable members including the fluid permeable membrane and a fluid permeable support positioned between the fluid permeable membrane and the second side of the fluid impermeable material.

5. The female urine collection device of claim 1, wherein the one or more permeable members include a plurality of permeable members including a fluid permeable membrane positioned on the female urine collection device to directly receive urine discharged from a female user and a fluid permeable support positioned between the fluid permeable membrane and the second side of the fluid impermeable material.

6. The female urine collection device of claim 1, wherein the fluid impermeable material has a bulbous portion protruding from the fluid impermeable material opposite to the opening and at least partially defines a reservoir disposed at least partially in the bulbous portion, wherein the reservoir is void of other materials.

7. The female urine collection device of claim 1, further comprising a conduit secured to the fluid impermeable material and in fluid communication with the chamber, the conduit being configured to provide fluid communication between a vacuum source and the chamber to remove the urine from the chamber.

8. A female urine collection device, comprising:
a fluid impermeable material having a lip defining an opening and a recessed region that is recessed from the lip and the opening, the fluid impermeable material defining a chamber therein, wherein the fluid impermeable material has a width of about 0.5 inch to about 1.0 inch and a length of about 0.5 inch to about 0.75 inch; and
one or more fluid permeable members disposed in the chamber at least proximate to the opening, at least a portion of the one or more fluid permeable members being positioned on the female urine collection device to directly receive urine discharged from a female user.

9. The female urine collection device of claim 8, wherein the fluid impermeable material is sized such that when the opening is covering a urethral opening of the female user, the perineum of the female user is generally free from coverage by the female urine collection device.

10. The female urine collection device of claim 8, wherein the one or more permeable members include at least a fluid permeable membrane, at least a portion of the fluid permeable membrane being positioned in the between the lip and the recessed region of the fluid impermeable material to directly receive the urine discharged from the female user.

11. The female urine collection device of claim 10, wherein the one or more permeable members include a plurality of permeable members including the fluid permeable membrane and a fluid permeable support positioned between the fluid permeable membrane and the recessed region of the fluid impermeable material.

12. The female urine collection device of claim 8, further comprising a conduit secured to the fluid impermeable material and in fluid communication with the chamber, the conduit being configured to provide fluid communication between a vacuum source and the chamber to remove the urine from the chamber.

13. A female urine collection device, comprising:
a fluid impermeable material having a lip defining an opening and a recessed region that is recessed from the lip and the opening, the fluid impermeable material defining a chamber therein; and
one or more fluid permeable members disposed in the chamber at least proximate to the opening, a portion of the one or more fluid permeable members being positioned on the female urine collection device to directly receive urine discharged from a female user,
wherein the lip of the fluid impermeable material and the portion of the one or more fluid permeable members positioned to directly receive the urine discharged from the female user define a contact region of the female urine collection device that is sized to substantially avoid contact with at least one of a clitoris, a vaginal opening, or a perineum of the female user when the opening is covering a urethral opening of the female user.

14. The female urine collection device of claim 13, further comprising a conduit secured to the fluid impermeable material and in fluid communication with the chamber, the conduit being configured to provide fluid communication between a vacuum source and the chamber to remove the urine from the chamber, wherein the contact region is configured such that when the vacuum source is activated, a suction force is limited to a targeted area of the female user including the urethral opening and excluding the at least one of the clitoris, the vaginal opening, or the perineum of the female user.

15. The female urine collection device of claim 13, wherein the contact region has a width of about 0.5 inch to about 1.0 inch and a length of about 0.5 inch to about 0.75 inch.

16. The female urine collection device of claim 14, wherein the one or more permeable members include at least a fluid permeable membrane, the contact region including at least a portion of the fluid permeable membrane.

17. The female urine collection device of claim 16, wherein the one or more permeable members include a plurality of permeable members including the fluid permeable membrane and a fluid permeable support positioned between the fluid permeable membrane and the recessed region of the fluid impermeable material.

18. A method of collecting urine from a female user, the method including:
positioning a contact region of a female urine collection device to cover a urethral opening of the female user without covering at least one of a clitoris, a vaginal opening, or a perineum of the female user, the contact region of the female urine collection device being defined by a lip of a fluid impermeable material of the female urine collection device defining an opening and a portion of one or more fluid permeable members of the female urine collection device, wherein fluid impermeable material includes a recessed region that is recessed from the lip and the opening and the one or more fluid permeable members are disposed in a chamber defined by the fluid impermeable material at least proximate to the opening;
directly receiving urine discharged from the female user on the portion of the one or more fluid permeable members defining the contact region and into the chamber; and
suctioning the urine from the chamber through a conduit in fluid communication with the chamber and a vacuum source without applying suction to the at least one of a clitoris, a vaginal opening, or a perineum of the female user.

19. The method of claim 18, suctioning is limited to a targeted area of the female user including the urethral opening and excluding the at least one of the clitoris, the vaginal opening, or the perineum of the female user.

20. The method of claim 18, wherein the contact region has a width of about 0.5 inch to about 1.0 inch and a length of about 0.5 inch to about 0.75 inch.
